(19) United States
(12) Patent Application Publication
(10) Pub. No.: US 2006/0276741 A1
(43) Pub. Date: Dec. 7, 2006

(54) Device and method for delivery of therapeutic agents to the dermis and epidermis

(76) Inventor: Julian L. Henley, Guilford, CT (US)

Correspondence Address: Michael G. Petit, Po Box 91929, Santa Barbara, CA, 93190-1929, US

(21) Appl. No.: 11/145,747

(22) Filed: Jun. 6, 2005

(51) Int. Cl. A61N 1/30

(52) U.S. Cl. 604/20

(57) ABSTRACT

Novel multichannel ionosonic devices are described for the treatment of disorders of the skin and nails. In a preferred embodiment, the ionosonic device includes a multichannel ionophoretic driver used in combination with a multichannel dispersion electrode integrated with ultrasonic elements on a single application electrode. The ionosonic device can be configured in a variety of shapes for the treatment of skin disorders through the intradermal delivery of one or more of a variety of therapeutic and modulating agents. The devices described herein deliver the therapeutic agents to the targeted diseased area, obviating the need for systemic ingestion and increasing the therapeutic efficacy for a variety of disorders of the skin and nails thereby sparing the individual from the toxic side effects of the therapeutic agent taken systemically.

### BACKGROUND OF THE INVENTION

1. Field of the Invention

The present invention relates to an intradermal drug delivery device and, more particularly, to a programmable multichannel iontophoresis electrode operable for delivering a therapeutic agent into the skin or nail.

2. Prior Art

The present inventor has disclosed and patented iontophoretic devices that has been shown to effectively treat herpetic infection of the mucocutanous junction. A number of studies demonstrate that a single channel iontophoretic device and electrode design according to previously specified parameters has been efficacious in treating, and rapidly attenuating, a herpetic skin infection in humans with a single 3-10 minute application of an antiviral agent. Studies have also demonstrated that such a device is able to drive a significantly greater amount of Acyclovir into the dermis as compared to topical application of the antiviral agent.

Such single channel therapeutic devices apply an ionophoretic current and carry the therapeutic agent (in this particular case, Acyclovir), into the lesion where the herpes virus is replicating. This approach has proven itself, after numerous studies, to be greatly efficacious in effectively treating the viral infection at the mucal cutaneous junction.

The technology described hereinbelow discloses novel intradermal drug delivery devices and discusses clinical applications therefor. In the context of the present invention, intradermal drug delivery devices are distinguished from prior art “transcutaneous” or “transdermal” drug delivery by specifically targeting layers of the dermis and epidermis for drug delivery. A multichannel ionophoretic driver for transdermal delivery of therapeutic agents and applications therefor have been previously patented by the present inventor. For example, a smokeless cigarette to help individuals cease smoking has been disclosed in U.S. Pat. No. 5,331,979 by the present inventor. The smokeless cigarette is based on the ionophoretic transport of nicotine through the mouthpiece of the device; the device having the general size and appearance of a cigarette. Upon contact with the lips, the facilitated transport of nicotine is activated and inhalation of smoke or other carcinogens is avoided. A wearable iontophoretic drug delivery device for substance abuse detoxification is disclosed in U.S. Pat. No. 5,538,503 to the present inventor.

A decade ago, animal studies demonstrated that ultrasound has a beneficial effect in improving penetration of skin in an animal model. The mechanism is thought to involve the disarray of the epidermal layer and perhaps dermal/epidermal microcavitation effects Several years ago, as disclosed in U.S. Pat. No. 5,658,247, the present inventor incorporated the multichannel ionophoretic technology with ultrasonic elements on a single application electrode for facilitated transdermal delivery.

The present inventor has previously disclosed electrodes and devices for the treatment of a variety of skin disorders based on the integrated use of a programmable multichannel (electromolecular) ionophoretic driver combined with ultrasonic elements which are integrated into a driver-electrode application system. As mentioned above, U.S. Pat. No. 5,538,503 by the present inventor discloses an apparatus for the programmable iontophoretic or iontophoretic-ultrasonic (ionosonic) transdermal delivery of medication across the skin or other biological membrane. In one embodiment, the programmed delivery of medicament is accompanied by an electrical stimulus for treating substance abuse. The apparatus can be adapted for large dermal area application or for a smaller area of application, depending on the choice of specific applicator electrode employed. In a preferred embodiment, the apparatus, disclosed in U.S. Pat. No. 5,658,247, comprises a multichannel iontophoretic applicator electrode. Multiple piezoelectric elements are mounted on the iontophoretic electrode. The combination of ultrasonic vibration and iontophoresis improves the penetration of medicament through the skin or mucous membrane underlying the electrode which can be programmably controlled by a CPU through the use of a EPROM. A wearable embodiment of an ionosonic or iontophoretic drug delivery system employing programmability may be used to produce response conditioning useful for the outpatient treatment of obesity, nicotine detoxification, and narcotic addiction detoxification.

The foregoing devices have been developed for the transdermal delivery of a medicament. There are many skin diseases and infections that may be treated by the intradermal application of a therapeutic or modulating agent. In addition to treating skin infections, which will be discussed below under DESCRIPTION OF THE PREFERRED EMBODIMENTS, there are proliferative disorders of the skin including early neoplastic and preneoplastic changes such as actinic keratosis or even frank basal cell carcinoma which may be treated by the intradermal delivery of a therapeutic agent. Widespread multiple dysplastic neoplastic lesions are now treated with 8-12 week application of topical antimetabolites such as 5FU. There are neoplastic and dysplastic disorders. There are also a number of hypoplastic and atrophic changes in the skin (many consider psoriasis a proliferative disorder) that may be treated by intradermal drug delivery. The current psoriasis treatments can be greatly enhanced and even help the patient self-treat if a device enabling increased local penetration of a therapeutic agent such as those medications that currently applied topically applied but limited in their efficacy by their lack of penetration into the psoriasis lesions.

Although wound healing is a natural process, a disturbance of the normal process forms a pathological condition that is of great clinical relevance. A clear-cut example is the post-burn wound contracture that requires extensive surgical intervention and grafting. The constant contracture of a scar creates distortion and interferes with normal function. Certain ethnic groups are also subject to keloids, which is another example of the healing process that continues to deposit new collagen when it is no longer needed for the healing process. The opposite polarity of this hyperactive wound healing is a deficient wound healing process. Wound dehiscence or insufficient collagen deposition where the wounds do not heal appropriately is an example of such a process often afflicting elderly, malnourished, immunocompromised patients and, for some, an idiopathic event. Other medical conditions may contribute to some of the aforesaid wound-healing disorders and there are a variety of agents that are designed to modulate this problem. Whereas as the current technology strives to deliver these agents topically or systemically, it would be desireable to provide a device that can deliver high doses of therapeutic agents to the dermal and the dermal junction which is the therapeutic area that needs to receive the agent in order to respond.

Yet a further category of skin disorders that may benefit from the provision of an intradermal drug delivery device is the anti-aging application. Although the aging process on the skin is a normal progression, society does not accept this process and there are a variety of products designed to ameliorate the aging process. There is a current need for a intradermal drug delivery device that potentiates existing agents significantly through the delivery into the dermis at high levels. Substances that may be efficacious in reducing the effects of aging are collagen deposition stimulants, vitamin C, anti-oxidants, chemotatic peptides and growth factors that may not only modulate new collagen formation but also wound healing.

Wound healing is a complex process modulated by many enzymes, peptides, and cellular activation. The sequence of these biochemical and cellular events is timed with the precision of an orchestra. Nevertheless, burns and other conditions create numerous challenges where intradermal drug delivery devices may prove beneficial. During the early stages of burn management the eschar has to be surgically removed (an exceptionally painful process), and wound dressings must be constantly maintained to prevent fluid loss and prevent infection. This requires extended hospital stay, multiple dressing changes, surgical debriedment and than surgical grafting. It is, therefor, desireable to provide an intradermal drug delivery device that can be applied to saturate the eschar with antibiotic/antifungal compounds so that the eschar can be used as a burn dressing until enzymatic separation occurs.

These secondary healing stages could further be modulated by providing an intradermal drug delivery device operable for driving enzymes, inteleukins, growth factor peptides and monoclonal antibodies targeted at specific healing stage enzymatic suppression into the skin at the burn site. Such technology may one day save significant costs and pain of burn management as many patient could be shifted from inpatient management to outpatient. A more challenging phase of bum wound management happens after successful grafting and closure of wounds. The scar tissue continues contract and creates a painful and disfiguring remainder of the primary burn. These patients are subjected to many years of scar excision and grafting in the attempt to return them to normal life with satisfactory limb function and acceptable appearance. The present inventor has conducted research in an animal model demonstrating a decrease of post injury contracture by use of systemic lathyrogenic agents. It would be desireable to provide an intradermal drug delivery device operable for driving a lathyrogenic agent directly into the contracting to achieve a significant benefit on this horrible sequela of burns without incurring the systemic toxicity when such agents are taken systemically. Since their action is on weakening the collagen, when taken systemically such agents can have life threatening side effects and therefore local wound therapeutic levels are seldom achieved.

A recent treatise on wound healing alludes to pharmacological modulators of wound healing including: (a) anti-inflammatory drugs such as aspirin, ibuprofen, naproxen and antihistaminics eg.diphenhydramine; (b) protein synthesis inhibitors including topical or intra-lesional steroids, colchicine, 5-flurouracil; (c) lathyrogenic agents such as BAPN, pencillamine or putrescine; (d) proteolytic enzyme synthesis stimulators include interleukin-1, calmodulin or protein kinase C inhibitors; and (e) calcium channel blockers such as verapamil or nifedipine. Accordingly, it is desireable to provide an intradermal drug delivery device to use for the benefit of burn treatment and wound healing modulation as well as the prevention of long term contracture.

There is currently a massive research effort to identify a new class of peptide therapeutic agents. Peptidic agents such as botox, epidermoid derived growth factors, chemo tactic agents, interleukins, etc have to be delivered by injection. Unfortunately such new products are peptides, and when taken systemically are not absorbed because they are broken down by stomach acid. Topically they have minimal penetration and are, therefore, not readily efficacious. It is desireable to provide an intradermal drug delivery device operable for driving small peptides directly into the diseased area in the skin.

The identification of particular therapeutic agents for the treatment of skin disorders begins by choosing molecular agents from pharmacological compounds, herbal compounds, or chemical compounds that demonstrate reasonable clinical efficacy. There are currently a number of available anti-inflammatory agents, such as steroids, antibiotic agents, anti-viral agents, anti-metabolite agents, immune modulating agents and anti-aging agents that modulate collagen metabolism in the dermis. Some of those compounds are patented and many are not. There is a need for an intradermal drug delivery device operable for delivering such agents to the desired diseased location (i.e., a targeted area of skin) without discomfort and in sufficient concentration to greatly enhance their therapeutic efficacy.

The practical limitations of existing topical ointments and formulations that contain therapeutic agents has been the minimal penetration of the epidermal layer and the fact that many topical agents do not sufficiently penetrate the dermis. Some existing therapeutic agents require 6-12 weeks of repeated application and general patient compliance is low. The topical applications are also limited by the fact that ointments, as soon as they evaporate, the penetration by passive diffusion stops. Researchers have modulated the depth of penetration through occlusion and the incorporation of various carrier molecules that facilitate intradermal transfer of the therapeutic agent. There is an ongoing need for an intradermal drug delivery device that can drive therapeutic agents into the dermis.

### SUMMARY

The intradermal drug delivery device of the present invention incorporates a multichannel iontophoresis electrode array on a single application electrode and includes one or more piezoelectric elements capable of generating vibratory ultrasonic fields to further enchance the penetration of therapeutic agents into the skin. In the preferred embodiment the device is programmable and includes a CPU and programming means such as an EPROM. The CPU and EPROM enable the operating parameters of each of the electrodes comprising the multichannel electrode to be independently controlled and changed.

Multichannel iontophoretic and inonosonic drug delivery devices disclosed herein have numerous clinical applications. A simple application of such a device is the treatment of a (very painful) herpetic infection affecting a large skin area and along a sensory nerve regional distribution referred to as Herpes Zoster or, more commonly, “Shingles.” Each channel of the multichannel device has a separate current limited driver circuit to prevent a runaway tunneling effect and make possible the application of therapeutic agent to a larger skin area yet retain control of an even distribution and penetration.

A related disease is viral warts and skin papillomas. Current modality involves surgery, laser burn, chemical burn, electrical burn and freezing. The present inventor has already successfully used this technology on several occasions with anecdotes of remarkable responses. For purposes of patenting this innovation such observations will suffice for purposes of wider acceptance such anecdotes will not be enough. Obviously, for many of the applications described herein, the FDA will require controlled studies to substantiate such claims before such approaches can become acceptable therapeutic options. It is the present inventor's intent to build and manufacture such devices that will open new therapeutic approaches to skin disorders whether they are a nuisance category, one of vanity, or serious life threatening nature.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a top plan view of one embodiment of a prior art multichannel iontophoretic applicator combined with several of the plurality of ultrasonic elements which can be used to treat large dermal areas.

FIG. 2 is a top, schematic, plan view of a the prior art multichannel iontophoretic-ultrasonic delivery system adapted for intradermal drug delivery.

FIG. 3 is a block circuit diagram of the prior art iontophoretic-ultrasonic (ionosonic) medicament applicator's electrical control circuit used in conjunction with above applicators either as a separate power and control unit or integrated into a single unit.

FIG. 4a is a palmar view of a glove having a plurality of iontophoresis applicator electrodes and return (neutral) electrodes disposed thereon and including a plurality of ultrasonic elements thereon.

FIG. 4b is a perspective view of a person employing the device of FIG. 3a to self-administer a cosmetic or therapeutic agent to the skin of the face thereafter the agent to be delivered to intradermal tissue.

FIG. 5 is a schematic view of a bootie having a plurality of iontophoresis electrodes and an ultrasonic electrode disposed thereon for delivering an antifungal agent between the toes.

FIG. 6 is a schematic view of a mask having a plurality of iontophoresis electrodes and ultrasonic elements disposed thereon for the intradermal delivery of a cosmetic or therapeutic agent to the skin of the face.

FIG. 7 is a schematic view of a bootie having a plurality of iontophoresis electrodes and an ultrasonic electrode disposed thereon for delivering an antifungal agent into one or more infected toenails.

### DESCRIPTION OF THE PREFERRED EMBODIMENTS

An abbreviated discussion of skin disorders is appropriate at this time, although a detailed discussion is not. There are a number of clinical disorders that afflict the dermal/epidermal layer that can be classified into inflammatory disorders exemplified by bacterial infection (e.g., Staphylococcus infection), viral infection (e.g., herpetic infestation), immuno reactive inflammatory disorders such as allergic welts, fungal infestation of skin and nails (e.g., monilia and onychomycosis (fungal infections of the nails and nail beds)) that chronically infects nails on both hands and feet. There also exist a variety of idiopathic inflammatory disorders such as localized vasculitis and number of rare blistering reactions, localized alopecia (loss of hair growth) and hypopigmentation (vitiligo). Even for idiopathic disorders there are topical agents that may have some benefit but they are currently severly restricted by lack of penetration. The multichannel and ionosonic technology described herein when combined with application electrode and existing or emerging therapeutic agent can vastly improve the efficacy of such treatments yet avoid systemic toxicity when a therapeutic agent is ingested in order to act regionally. Such new treatment approaches would open therapeutic options for existing compounds whose ingestion toxicities have not been tested as they will be used topically and regionally with minimal systemic absorption.

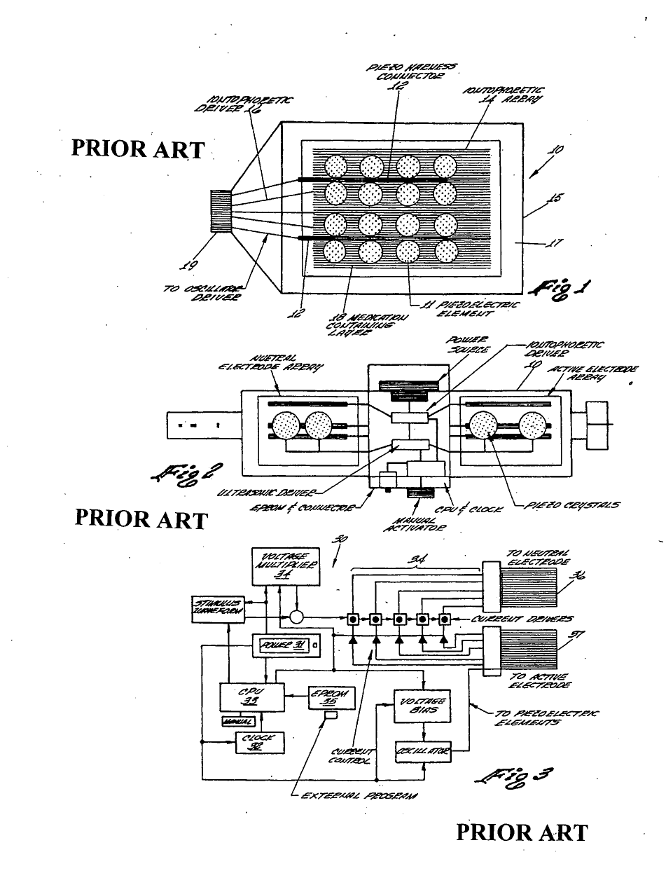

A prior art multichannel ionosonic applicator, generally indicated at the numeral 10, is shown in FIG. 1. The prior art applicator 10 includes a working electrode 14 which forms a closed circuit through the patient's body when current passes therethrough which promotes the penetration or absorption of an ionic medicament contained in a layer 18 adjacent to and in electrical communication with the working electrode 14. The polarity of the working electrode 14 is selected based upon the polarity of the medicament to be administered. The electrode 10 preferably comprises a flexible sheet or film forming a conductive matrix 15 having a current distributing conductive layer, such as a metallic foil, a conductive rubber or resin film, carbon film or other conductive coating or electro-dispersive material. The conductive matrix 15 is flexible so that it may be contoured to the body area on which it is placed and still cover a relatively wide area. Matrix 15 has a medicament carrying layer 18 attached to it, such as by an adhesive. The medicament carrying layer 18 is preferably formed from a porous material about ¼ of an inch thick which can be a honeycombed sponge-like material with vertical cells to minimize cross flow or lateral dispersion of the medicament. The grounding electrode (not shown) employed with the multichannel electrode 14 must also cover an area of skin which is similar in size to the area covered by electrode 14.

A ribbon connector (not shown in FIG. 1) connects an electrical power source (again not shown in FIG. 1) to the multichannel electrode 14 and delivers the electrical current by means of the multiconnectors 19 to the lead wires 16 that form the individual electrically conductive channels in the conductive matrix 15. Since the material of construction is flexible, the electrode 10 may be folded over a rigid supporting substrate above the connectors 19 to insure that a good electrical connection is made with the ribbon connector. Each channel in the iontophoretic array 14 preferably carries no more than 1 milliamp. The amount of current that flows to each channel is controlled by the control circuit (shown in FIG. 3) to prevent a tunneling effect from occurring. This prevents the flow of current along the path of least resistance through a lesion or skin rupture, for example, resulting in a burn to the patient at that location. The multichannel electrode 14 can employ a circuit pattern etched such as by laser or photoetching onto, for example, a metal coated Mylar RTM plastic sheet with each channel isolated to facilitate dispersion over a broad surface area.

Each channel formed by the lead wires 16 can be electrically driven simultaneously or in a sequential multiplex fashion. The use of simultaneous or parallel electrical current to each lead wire 16 in the array 14 would be employed, for example, in the application of medicament to burns where a wide area of dispersion is required. The iontosonic applicator greatly improves the skin penetration by the medicament to actively deliver the medicament to either a wide regional area of the skin or to a specific lesion on the skin.

Ultrasonic elements 11 made of piezoelectric crystal elements are mounted on this flexible electrode by means of a suitable adhesive such as Silastic® brand of silicone adhesive. Driving oscillator connections 12 to the crystals can be photoetched onto a polymer sheet (e.g.; metalized Mylar®) with perforations on the sheet which facilitate mounting of the ultrasonic elements. This electrode can be effective in moving Insulin across skin, as well as antibiotics, antifungal, anti-inflammatory, blood pressure medication and cardiotropic drugs; either as direct drive, logic control timer drive or more elegantly as biofeedback control configuration. It is also effective in the treatment of wide field dermatological conditions, such as eczema, psoriasis and acne. It is also effective for ionic retention of skin hydrating media to facilitate skin hydration in cosmetic applications and in dermal exfoliation to drive medication into the skin in order to inflame the skin and cause the peeling of the external skin layer to stimulate reformation of collagen and collagen growth factors. The ionosonic applicator may also prove useful for driving Minoxidil® or related compounds into the scalp to enhance hair growth and/or ameliorate baldness. The construction of ultrasonic elements can be piezo-electric crystals, ceramics or distributed segments of Kynar® PVDF piezo film.

The open-celled sponge-like material in the medicament carrying layer 18 should be inert to the medicament or treatment agent being employed, as well as being noncorrosive and stable. Suitable materials include plastic pads, such as polyethylene, paper or cotton, porous ceramics, open-celled porous polytetrafluoro ethylene, other inert plastics, and open-celled silicone rubber, preferably with vertically aligned medicament-containing cells or tubes.

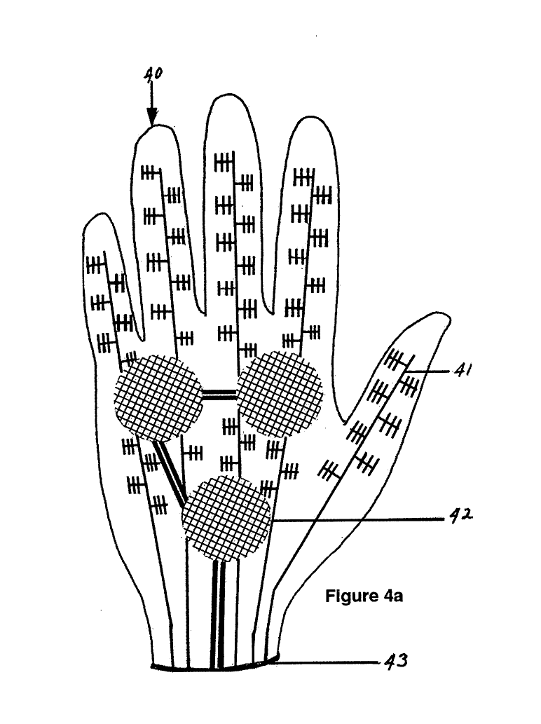

FIG. 3 shows a block circuit diagram of the iontophoretic medicator electrical control circuit suitable for use with the multichannel ionosonic applicator of FIG. 1 and the miniaturized ionosonic applicator diagrammed in FIG. 2. The control circuit, generally indicated at 30, may be either integrated with the applicator electrode, as shown in FIG. 2, or boxed separately and including connection means adapted to electrically connect to the applicator to provide power to drive the applicator as shown in FIG. 1. The control circuit is equipped with a power source 31 which may be either a battery or an isolated wall source.

The control box 30 is provided with a clock-operated timer switch 32 to preset the length of iontophoretic treatment mediated by the integral CPU. Once the length of time has been selected, a voltage multiplier is utilized to provide the current to iontophoretically drive the medicament into the patient's skin. The current is set and administered until the end of the treatment period. When the clock 32 signals the end of the treatment period, the electrical current to the electrode 10 is gradually terminated by a ramping down of the current to the patient to avoid abrupt change. Ribbon cable (not shown) provides a flexible connection to the multichannel neutral and active electrodes as indicated in FIG. 3, as well as delivering oscillator power for the piezoelectric crystals 11 mounted on the applicator electrode 10. Internal circuit board controls allow for frequency adjustment, adjustment of maximum current per iontophoretic channel (not to exceed 0.6 to 1.2 ma range),and internal control that will shut down any iontophoretic channel electrically performing outside a “normal” range of encountered biological impedance.

With further reference to FIG. 3, the block circuit diagram of the large area iontophoretic medicator control circuit is shown which can be employed with the multichannel ionosonic applicator of FIG. 2. An isolated current loop generator is employed to feed current to the individual channels in the multichannel electrode via the plurality of individual current loops. Each current loop drives one band or channel in the multichannel electrode. It has been found that 0.6 milliamps current flowing to each channel used within a wide field dispersion grounding electrode, such as that shown in FIG. 1, provides a safe level for operating the ionosonic device. This level of current avoids the tunnelling effect of current flowing along the path of least resistance and concentrating in, for example, a lesion or skin rupture, resulting in a burn to the patient. This permits current to be distributed over the large area of the multichannel electrode to drive medicament through a patient's skin over a large dermal area. Depending Upon the electrode configuration, this current level can vary from about 0.1 to about 1.2 milliamps. The novel introduction of distributed ultrasonic piezoelectric elements combined with the iontophoretic multi electrodes described above greatly enhances the rate of penetration of many molecules. The use of ionosonic applications to administer insulin transdermally now becomes feasible.

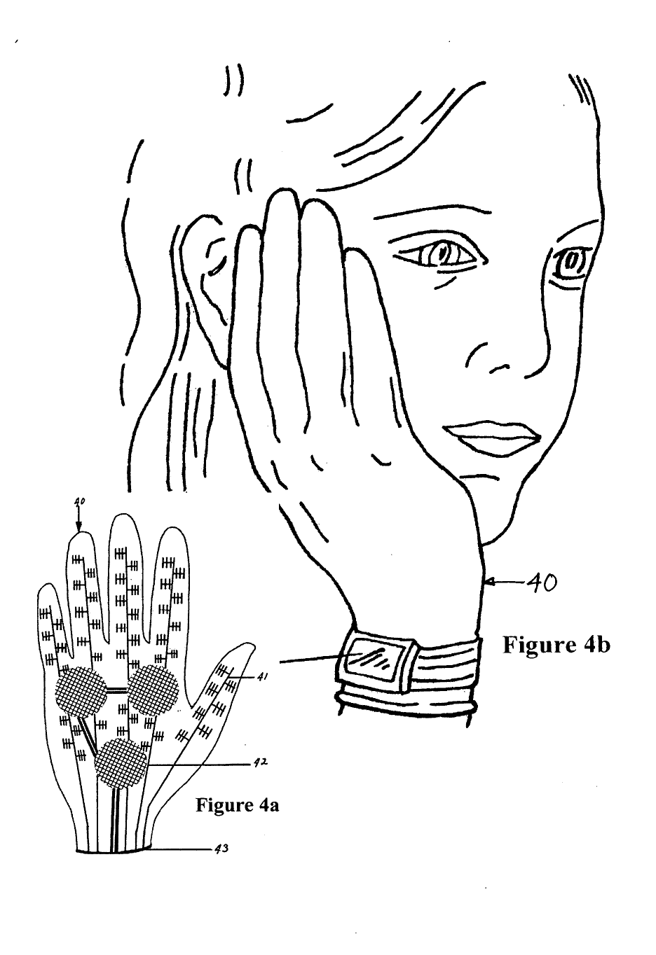

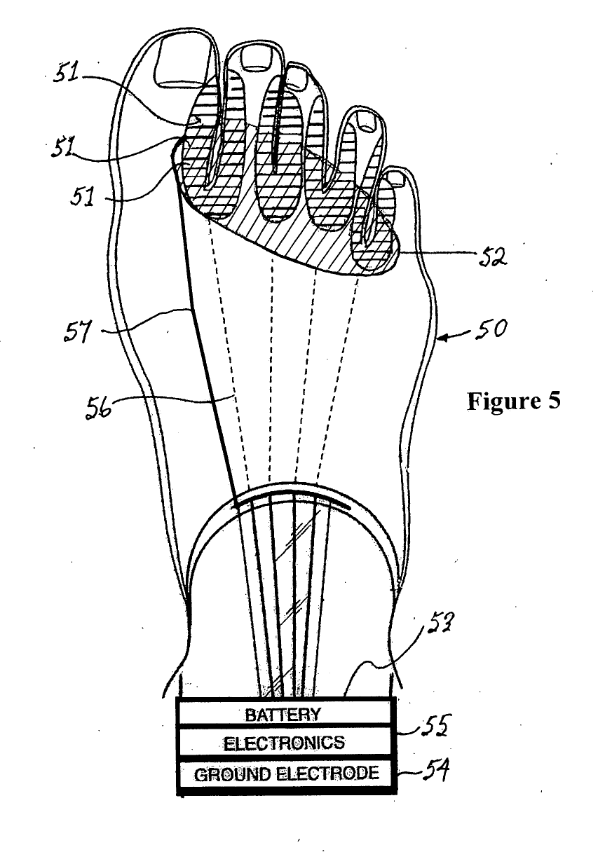

The multichannel ionosonic device described above has many applications wherein it is desired to deliver a therapeutic agent only into the epidermis and dermis (i.e., intradermally). FIG. 4a is a palmar view of a glove 40 having a plurality of iontophoresis applicator electrodes 41 and return (neutral) electrodes disposed thereon and including a plurality of ultrasonic elements 42 disposed thereon. The electrodes 41 and piezoelectric elements 42 are in electrical connection with a power source (not shown in FIG. 4a) by a connector harness 43 affixed to the glove. FIG. 4b is a perspective view of a person wearing an ionosonic glove applicator 40 of FIG. 4a and employing the device 40 to self-administer a cosmetic or therapeutic agent to the skin of the face thereafter the agent to be delivered to intradermal tissue.

FIG. 5 is a schematic view of an ionosonic bootie 50 having a plurality of iontophoresis electrodes 51 and an ultrasonic electrode 52 disposed thereon for delivering an antifungal agent into the skin of the toes. The bootie 50 includes a power source 53, a grounded electrode 54 which is in electrical communication with the patients skin distal from the toes, and an electronic circuit 55 which is preferably programmable and operable for providing a driving current to the iontophoresis electrodes 51 and piezo element 52 through conductors 56 and 57 respectively.

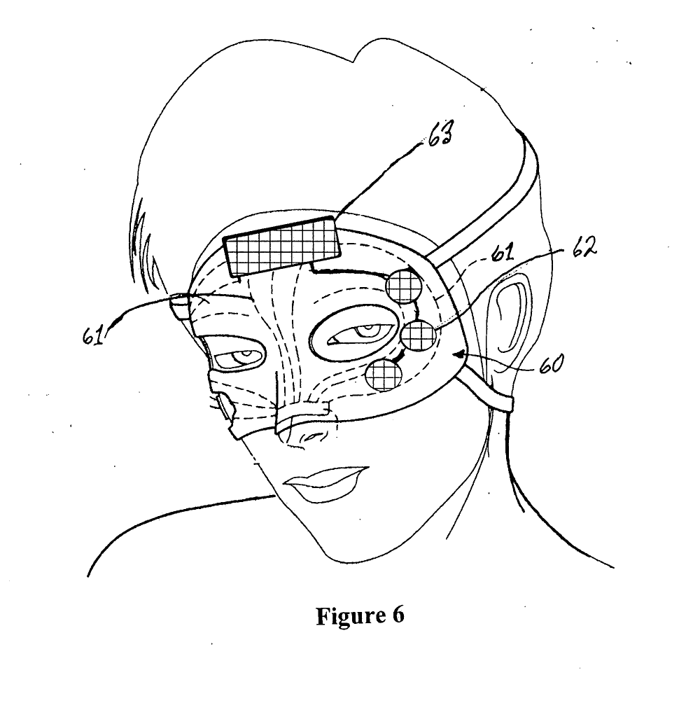

FIG. 6 is a schematic view of an ionosonic mask 60 similar in operation to the ionosonic bootie 50 and having a plurality of iontophoresis electrodes 61 and ultrasonic elements 62 disposed thereon for the intradermal delivery of a cosmetic or therapeutic agent to the skin of the face. The mask 60 includes a driver 63 operable for providing a driver current to the iontophoresis electrodes 61 and the piezoelectric ultrasonic elements 62. The mask 60 is useful for the intradermal delivery of cosmetic/therapeutic agents for the treatment of wrinkles.

A particularly recalcitrant medical condition that is benign but unsightly is onychomycosis. Onychomycosis, also called ringworm of the nails, or tinea unguium, is a fungus infection of the nails causing thickening, roughness and splitting. The infection is caused by various fungal species including Trichophyton rubrum, Trichophyton tonsurans, Epidermophyton floccosum and Trichophyton mentagrophytes. Once these microorganisms establish themselves within or under a nail, eradication with current over-the-counter (OTC) antifungal agents is difficult, costly and time consuming and recurrences of the disease can be expected. Antifungal agents that are formulated for topical application, such as undecylenic acid, lotrimin and tolnaftate, are effective for treating fungal infections of the skin but, as presently formulated and administered, are ineffective for treating fungal infections of the nail.

Current treatment regimens for onychomycosis include prolonged and sustained application of topical fungicidal creams and/or solutions directly to the infected nail(s) and/or systemic treatment with antifungal drugs such as griseoflulvin, terbinafine, and itraconazole. Some of the systemic treatments have undesirable side effects such as nausea, headache, photosensitivity, gastrointestinal intolerance, elevated liver enzymes, and undesirable drug interactions, making the value of the treatment questionable.

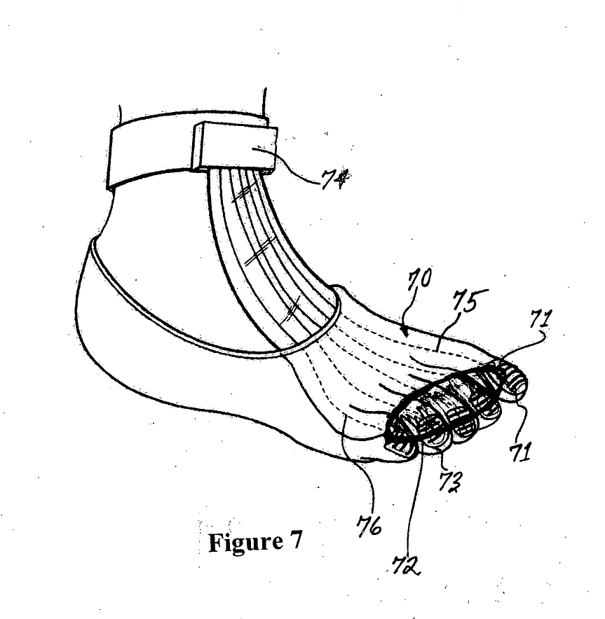

FIG. 7 is a schematic view of an ionosonic bootie 70 having a plurality of iontophoresis electrodes 71 and at least one ultrasonic electrode 72 disposed on an inner skin-contacting surface thereof that is operable for delivering an antifungal agent into one or more infected toenails 73. The bootie 70 includes a driver 74 comprising a power source such as a battery and an electronic controller circuit, preferably programmable, operable for conducting a driving current to the iontophoresis electrodes 71 and the ultrasonic piezo element(s) 72 through electrical conductors 75 and 76 respectively. The antifungal agent may be applied directly to the infected nail prior to donning the boot or contained within a reservoir comprising the iontophoresis electrodes.

The embodiments of the multichannel ionosonic intradermal drug delivery device discussed above are wearable and conform to the contour of the area of the infected or deformed skin and/or nail to be treated. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What I claim is:

1. An ionosonic intradermal drug delivery device adapted to releasably attach to a person's body so that a skin-contacting surface of said device is adjacent to a targeted portion of the person's skin or nail, said device being operable for ionosonically driving a medicament across said skin-contacting surface of said device into said targeted portion of the person's skin or nail, said device comprising, in combination: (a) a medicament carrying layer in fluid communication with said skin-contacting surface of said device comprising a porous sheet impregnated with a medicament containing fluid; (b) an iontophoresis electrode in electrical communication with said medicament carrying layer providing means for iontophoretically driving said medicament into the targeted portion of the person's skin or nail(s) wherein said iontophoresis electrode comprises a plurality of electrode channels, each electrode channel of said plurality of electrode channels being electrically isolated from other electrode channels, said plurality of electrode channels having electrical connection means connected thereto adapted for simultaneous electrical connection of selected electrode channels to the same or different current sources, said each electrode channel being in electrical communication with said medicament carrying layer; and (c) at least one piezoelectric element affixed to said iontophoresis electrode and overlying at least one of said iontophoresis electrode channels.

2. The ionosonic intradermal drug delivery device according to claim 1 wherein said at least one medicament is operable for treating or preventing microbial infection of skin or nail.

3. The ionosonic intradermal drug delivery device according to claim 1 wherein said at least one medicament is operable for treating a wound.

4. The ionosonic intradermal drug delivery device according to claim 1 wherein said at least one medicament is operable for treating or preventing acne.

5. The ionosonic intradermal drug delivery device according to claim 1 wherein said at least one medicament is operable for treating psoriasis.

6. The ionosonic intradermal drug delivery device according to claim 1 wherein said at least one medicament is operable for treating or preventing eczema or contact dermatitis or atopic dermatitis.

7. The ionosonic intradermal drug delivery device according to claim 1 wherein said at least one medicament is operable for treating or preventing onychomycosis.

8. The ionosonic intradermal drug delivery device according to claim 1 wherein said at least one medicament is operable for treating actinic keratoses.

9. The ionosonic intradermal drug delivery device according to claim 1 wherein delivery of the at least one medicament is operable for treating skin cancer.

10. The ionosonic intradermal drug delivery device according to claim 1 wherein said at least one medicament is an anesthetic.

11. The ionosonic intradermal drug delivery device according to claim 1 wherein said at least one medicament is operable for treating an infection by a human papilloma virus.

12. A method for the intradermal self-administration of a medicament into a targeted portion of the skin or nail of an individual comprising the steps of: (a) presenting an ionosonic intradermal drug delivery device in accordance with claim 1; (b) impregnating said porous sheet with a medicament and placing said porous sheet in contact with said targeted portion of the individual's skin or nails; and (c) causing an electrical current to flow through said electrode channels of said iontophoresis electrode in an amount sufficient to drive the medicament into the targeted portion of the skin or nail.
